(19) United States
(12) Patent Application Publication
(10) Pub. No.: US 2007/0059263 A1
(43) Pub. Date: Mar. 15, 2007

(54) Solid skin care composition comprising multiple layers

(76) Inventors: Toshiya Taniguchi, Kobe (JP); Takashi Sako, Kobe (JP); Yoko Fujiwara, Himeji (JP); Kiyohiko Fujii, Ota-ku (JP); Nobuo Watanabe, Ota-ku (JP)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/517,981

(22) Filed: Sep. 8, 2006

Related U.S. Application Data
(60) Provisional application No. 60/715,687, filed on Sep. 9, 2005.

(51) Int. Cl. A61K 8/89

(52) U.S. Cl. 424/63; 424/401

(57) ABSTRACT

The present application relates to a solid skin care composition comprising: (a) a first layer which is solid at 45° C. and which is a water-in-oil emulsion or an oil-in-water emulsion; and (b) a second layer which is solid at 45° C. and which is an oil dispersion comprising a benefit agent; and wherein the first layer and the second layer are provided in the same package in a manner such that the first layer and the second layer can be simultaneously applied.

### CROSS REFERENCE TO RELATED APPLICATION

This application claims the benefit of U.S. Provisional Application No. 60/715,687 filed on Sep. 9, 2005.

### FIELD OF THE INVENTION

The present invention relates to a solid skin care composition comprising multiple layers. Specifically, the present invention relates to solid skin care compositions comprising multiple layers each made of different compositions providing unique characteristic benefits. The characteristic benefits would not be achieved to the extent when provided in separate phases, if the multiple layers were mixed together and provided as a single composition. The compositions of the present invention are particularly useful for cosmetic foundation products.

### BACKGROUND OF THE INVENTION

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders or anhydrous oil and wax compositions. Emulsion-type foundations in the form of liquid are suitable in that they provide moisturizing effects by the water and water-soluble skin treatment agents incorporated. These liquid form foundations, however, are less convenient to use and carry for the consumer. On the other hand, solid foundations packaged in compacts are suitable for use by the consumer, however, are typically less efficient than liquid form foundations in terms of moisturizing the skin and coverage of the skin.

Foundation compositions in the form of solid, yet emulsion have been suggested. Such solid emulsion foundations aim to address the drawbacks of conventional liquid form foundations and solid foundations. These foundations can be filled in a wide variety of packaging, including compacts, and is increasing popularity among consumers. References which disclose such foundation compositions include Japanese patent publications A-2-88511, A-3-261707, A-7-267819, A-11-209243, U.S. Pat. No. 5,362,482, and PCT publication WO 01/91704.

Recently, consumers have become to seek various performances and benefits in foundation products, such as wear, spreadability, fit to the skin, blending into the skin, coverage, long lasting, oil shine control, UV protection, Further, different consumer segments may seek different types of performance, such as moisturizing feel against light feel, and natural look against lustrous finish. To achieve these benefits, foundation formulations must accommodate various components which, depending on their physical and chemical properties, may be difficult to formulate into a single product. For example, oil shine control is a highly desirable function for a foundation product. However, incorporation of oil absorbing powders at a high level will render the formulation to have a very heavy application feel with poor spreadability. Incorporation at a high level may also make the emulsion unstable.

On the other hand, cosmetic compositions comprising multiple layers or phases are known in the prior art. These products are usually provided in the phase types of cream, gel, or paste and are usually focusing on the distinctness of the color of each layer. For example, U.S. Pat. No. 4,980,155 to Revlon, Inc. discloses a two phase cosmetic composition comprising a color phase composition and a gel phase composition. WO2004/105708 to Gamma Croma S.P.A. discloses a multicolor cosmetic product with solid consistence that comprises two or more cosmetic products of different colors. JP Patent Application Publication No. 1999-269025 to Noevir Co., Ltd. discloses a double-layered stick-shaped cosmetic product comprising an oil-based stick-shaped composition and a water-based stick-shaped composition. JP Patent Application Publication No. 2002-97112 discloses a solid cosmetic composition having mutually different colors and the manufacturing process for the same. None of them discloses a multi-layered skin care composition which is in the form of solid water-in-oil or oil-in-water emulsion and oil dispersion in ambient temperature.

Based on the foregoing, there is a need for a solid skin care composition which provides more than one benefit rendered by components which are difficult to formulate into a single composition. Specifically for cosmetic foundation products, there is a need for a solid composition which provides good spreadability, wear as well as other skin benefits in one product.

None of the existing art provides all of the advantages and benefits of the present invention.

### SUMMARY OF THE INVENTION

The present invention is directed to a multiple layer solid skin care composition comprising:

(a) a first layer which is solid at 45° C. and which is a water-in-oil emulsion or an oil-in-water emulsion; and

(b) a second layer which is solid at 45° C. and which is an oil dispersion; wherein the first layer and the second layer are provided in the same package in a manner such that the first layer and the second layer can be simultaneously applied. By providing multiple layers of compositions in a manner such that they can be simultaneously applied, the overall composition provides benefits characteristic of each layer, which benefit(s) would otherwise be compromised or deteriorate other performance, if they were combined into one composition.

The present invention is suitable for any skin care composition in solid form, such as cosmetic foundation, blusher, sunscreen, eyeshadow, lipstick, antiperspirant stick, dermal pharmaceutical ointment, and others. One particularly preferred embodiment for the present invention is a cosmetic foundation made of multiple layers that are visibly distinct.

In another aspect, the present application relates to the manufacture process for a multilayer skin care composition comprising the steps of:

(a) providing the first layer composition and the second layer composition in fluid state in isolated vessels;

(b) separately dispensing the first layer composition by a first nozzle and the second layer composition by a second nozzle into a same package while keeping the temperature of the first layer composition and second layer composition between 55° C. and 90° C., preferably between 60° C. and 75° C.; and

(c) allowing the transferred first layer and second layer to solidify in the package.

These and other features, aspects, and advantages of the present invention will become evident to those skilled in the art from a reading of the present disclosure with the appended claims.

### BRIEF DESCRIPTION OF THE DRAWINGS

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is a schematic view of a preferred embodiment of the process of the present invention.

FIG. 2 is a sectional view of FIG. 1 taken at line A-A′.

FIG. 3(a)-(d) are schematic views of preferred embodiments of the process of the present invention focusing on the filling step.

FIG. 4(i)-(vii) are schematic views of preferred embodiments of the visible appearance of the present composition.

FIG. 5 is a diagram showing the preferred range of viscosity difference and density difference between the compositions of the first layer and the second layer of the present invention.

### DETAILED DESCRIPTION OF THE INVENTION

While the specification concludes with claims particularly pointing out and distinctly claiming the invention, it is believed that the present invention will be better understood from the following description.

All percentages, parts and ratios as used herein are by weight of the composition of each layer, unless otherwise specified. All such weights as they pertain to listed ingredients are based on the active level and, therefore do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

First Layer And Second Layer

The composition of the present invention comprises multiple layers, namely at least a first layer and a second layer. By providing multiple layers of compositions in a manner such that they can be simultaneously applied, the overall composition provides benefits characteristic of each layer, which benefit(s) would otherwise be compromised or deteriorate other performance, if they were combined into one composition. While any number of layers can be included in the overall composition, an overall composition having two layers is focused in the discussion herein.

The first and second layers are of different composition, and are designed to provide different benefits based on at least one benefit agent included in either of the layers. For convenience, the layer comprising such benefit agent is called the second layer; however, this does not require that the first layer is devoid of a benefit agent. The first and second layers may comprise different benefit agents, different combination of benefit agents, or different concentrations of the same benefit agent. In the context of the present invention, a “benefit agent” is a component which provides a particular skin benefit characteristic of the usage of the skin care product. Herein, skin care benefit may include benefits related to appearance or make-up of the skin. Typically, a certain benefit agent included in the second layer is less compatible with a certain component included in the first layer, or a certain benefit agent in the second layer deteriorates performance of the overall composition when the first and second layers are combined into one composition.

For example, oil shine control is a favorable characteristic of a cosmetic foundation product. Inclusion of oil absorbing powders as a benefit agent provides the oil shine control benefit. However, inclusion of oil absorbing powders at an effective level may also provide unfavorable spreadability performance. In the present invention, by including the oil absorbing powders mainly in the second layer composition, and providing the first and second layers in a manner such that they can be simultaneously applied on the skin, the spreadability and the powdery feeling of the overall composition is significantly improved. In other words, the oil shine control benefit of the second layer composition and the overall foundation benefit of the first layer composition can both be provided in the overall composition of the present invention.

The first and second layers of the present invention are solid at room temperature, thus do not, or only slightly dissolve or mingle with each other during storage, and after each use. The first and second layers are provided in a manner that allows the user to simultaneously apply both layers to the skin. A suitable way is to provide both layers in the same primary package, for example a pan, jar, or stick applicator. The primary package may accompany a suitable applicator, such as a sponge or brush. Preferably, the first and second layers are formulated such that they exhibit a similar rheology profile when receiving pressure/heat from the finger or applicator upon use.

The first and second layers can be provided in any ratio as necessary for providing the target benefit(s). Preferably, the first and second layers are provided in a weight ratio of from about 1:99 to about 99:1, more preferably from about 1:9 to about 9:1. The first and second layers are preferably visibly distinct, so that the different benefits/characteristics of the layers are communicated to the user. A colorant may be suitably included in at least one of the first or second layers for making the layers visibly distinct.

Phase Type and Formulation of the First Layer and Second Layer

In the present invention, the composition of the first layer takes the phase type of water-in-oil emulsion or oil-in-water emulsion. The composition of the second layer takes the phase type of oil dispersion. Water-in-oil emulsions and oil-in-water emulsions are useful for providing good application feel to the skin, while also being able to encompass oil soluble and water soluble components, and further leaving a fresh and cool feeling after the water and/or volatile oils is evaporated. Oil dispersions are useful for providing good application feel to the skin, while also being able to encompass high level of powders to increase powdery feeling and oily shine control.

In one highly preferred embodiment, the present composition is a cosmetic foundation.

When the first layer is a water-in-oil emulsion, the first layer composition preferably comprises the following components:

(a) from about 10% to about 50%, preferably from about 15% to about 35% of a volatile silicone oil;

(b) from about 0.5% to about 20%, preferably from about 1% to about 15% of a non-volatile oil;

(c) from about 5% to about 45%, preferably from about 15% to about 30% of a pigment powder;

(d) from about 1% to about 10%, preferably from about 2% to about 5% of a solid wax;

(e) from about 0.5% to about 5%, preferably from about 1% to about 4% of a lipophilic surfactant; and

(f) an amount of water, such that the total level of the volatile silicone oil and water is more than about 40%, preferably from about 10% to about 35% of water.

When the first layer is an oil-in-water emulsion, the first layer composition preferably comprises the following components:

(a) from about 20% to about 60%, preferably from about 30% to about 50% of water;

(b) from about 0.1% to about 4%, preferably from about 0.3% to about 2% of a hydrophilic surfactant;

(c) from about 5% to about 40%, preferably from about 10% to about 30% of a pigment powder;

(d) from about 1% to about 20%, preferably from about 5% to about 15% of a non-volatile oil.

(e) from about 1% to about 30%, preferably from about 5% to 20% of a volatile silicone oil; and

(f) from about 1% to about 15%, preferably from about 4% to about 10% of fatty compound or fatty acid salts.

The second layer composition which is oil dispersion preferably comprises the following components:

(a) from about 10% to about 80%, preferably from about 20% to about 70% of a volatile silicone oil;

(b) from about 1% to about 40%, preferably from about 5% to about 25% of a non-volatile oil;

(c) from about 1% to about 10%, preferably from about 2% to about 7% of a solid wax; and

(d) optionally a pigment powder, when present, from about 1% to about 70%, preferably from about 5% to about 50% of a pigment powder.

The second layer further comprises at least one benefit agent selected from oil absorbing powders, sebum solidifying powders, film forming polymers, soft focus agents and mixtures thereof. Compositions of each layer are formulated to have a viscosity value of from about 100 mPas to about 10,000 mPas, preferably from about 500 mPas to about 3,000 mPas when brought to a temperature of between about 55° C. and about 90° C.

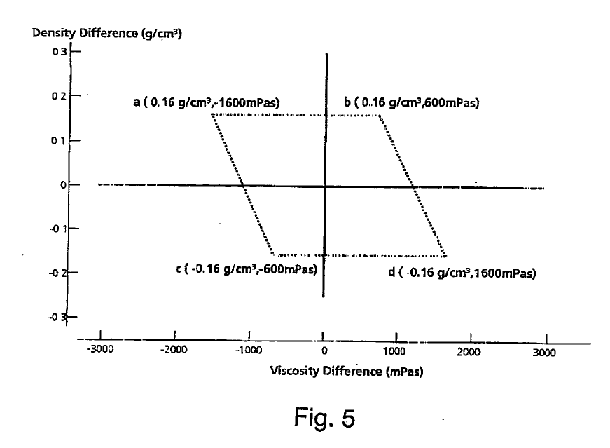

As will be explained in detail in the following context, the compositions of each layer are formulated and formed separately. Once formulated and formed, each respective layer can be combined during the packaging process by dispensing the respective layers simultaneously into a primary package, such as a pan or the like in a swirl, a spiral, a rod, a flower or the like configuration. In order to keep the two layers separate from each other for a prolonged period, it is preferably that each layer is formulated to keep the viscosity difference and density difference between the compositions of each layer in the area defined by the four points of a(0.16 g/cm³, −1600 mPas), b(0.16 g/cm³, 600 mPas), c(−0.16 g/cm³, −600 mPas) and d(−0.16 g/cm³, 1600 mPas) as shown in the diagram of FIG. 5. The method used to adjust the density and viscosity of the composition of each layer is known to those skilled in the art. It has been found that when the density difference and viscosity difference between the compositions of each layer are within the preferred area, the two layers exhibit favorable physical stability over a period of time. To make the two layers visually distinctive, a preferred way is to use different species and/or level of pigment in the composition of each layer. Details of the species and levels of the components contained in each layer are provided as follows.

Benefit Agent

The composition of the present invention comprises a benefit agent which provides a particular skin benefit characteristic of the usage of the skin care product. Herein, skin care benefit may include benefits related to appearance or make-up of the skin.

In a cosmetic composition embodiment, including but not limited to cosmetic foundation, blusher, sunscreen, eyeshadow, lipstick, the benefit agent is selected from the group consisting of oil absorbing powder, sebum solidifying powder, film forming polymer, soft focus agent and mixtures thereof.

In an antiperspirant embodiment, the benefit agent is an antiperspirant active.

Oil Absorbing Powder

Oil absorbing powder is a pigment that is particularly effective in absorbing oil, and thereby can be included in the present composition for absorbing excessive sebum from the skin. Specifically, the oil absorbing pigment herein has an oil absorbency of at least about 100 ml/100 g, preferably at least about 200 ml/100 g. Oil absorbency is a unit well known to the artisan, and which can be measured via: JIS K5101 No.21 “Test Method for Oil Absorbency Level”.

Oil absorbing powder useful herein includes spherical silica and methyl methacrylate copolymer. Commercially available oil absorbing powders useful herein include spherical silica with tradename SI-SILDEX H-52 available from Miyoshi Kasei, Inc. having an oil absorbency of more than 200 ml/100 g, vinyl dimethicone/methicone silsesquioxane crosspolymer with tradename KSP-100 and KSP-101 available from ShinEtsu Chemical having an oil absorbency of more than 200 ml/100 g, hardened polyorgano siloxane elastomers with tradename TREFIL E-506C available from Dow Coming having an oil absorbency of more than 100 ml/100 g, and methyl methacrylate copolymer with tradename SA-GMP-0820 available from GANZ Chemical and surface treated by Miyoshi Kasei, Inc. having an oil absorbency of more than 100 ml/100 g. Typically, inclusion of oil absorbing powders for oil shine control may provide a composition with unfavorable spreadability performance. However, in the present invention, by including oil absorbing powders mainly in the second layer, the unfavorable spreadability performance can be improved. In a preferred example, the content level of an oil absorbing powder in the second layer is from about 1% to about 20%, more preferably from about 2% to about 15%.

Sebum Solidifying Powder

Sebum solidifying powder useful herein includes those comprising a base substance which is coated with low crystalline zinc oxide, amorphous zinc oxide, or mixtures thereof, wherein the zinc oxide is from about 15% to about 25% by weight of the sebum solidifying powder. The base substance may be any organic or inorganic substances that are useful for cosmetic use, including those listed below under “Pigment Powder Component”. The sebum solidifying powder herein can be suitably made according to the methods disclosed in US 2002/0031534A1, herein incorporated by reference. The sebum solidifying powders may be surface treated. The sebum solidifying powder useful herein has the ability to solidify sebum, i.e., are effective in adsorbing free fatty acid, diglyceride, and triglyceride, and solidifying them by forming zinc salts thereof, such that a film is formed within about 30 minutes. Moreover, the originally glossy sebum changes appearance into a matte film. Such capability can be distinguished from other oil absorbing powders, which are not selective in the type of oil to the absorbed, and do not form a film after absorbing an oil, thus may leave glossy gels and pastes after absorbing the sebum. Change in appearance provides a noticeable signal to the user that sebum has been controlled. Sebum solidifying effect may be conveniently measured by mixing a certain amount of powder with a certain amount of artificial sebum, mixing for a certain period of time, and allowing standing until solidified or showing matte appearance. The time taken for the mixture to solidify or to change appearance is recorded. The shorter the time taken to solidify or change appearance, the higher the solidifying effect is of the powder.

Commercially available sebum solidifying powder useful herein include mica coated with hydroxyapatite, 20% zinc oxide with tradename PLV-20, and the same powder surface treated with methicone with tradename SI-PLV-20, both available from Miyoshi Kasei, Inc. Typically, inclusion of sebum solidifying powders for oil shine control may provide a composition with unfavorable spreadability performance. However, in the present invention, by including sebum solidifying powders mainly in the second layer, the unfavorable spreadability performance can be improved. In a preferred example, the content level of sebum solidifying powder in the second layer is from about 0.2% to about 30%, preferably from about 1% to about 15%.

Film Forming Polymer

Film forming polymer is useful for imparting wear and/or transfer resistant properties to a cosmetic product. Preferred polymers form a non-tacky film which is removable with water used with cleansers such as soap.

Examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E.S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, published on Oct. 31 1996 WO97/17058, published on May 15, 1997 and U.S. Pat. No. 5,505,937 issued to Castrogiovanni et al. on Apr. 9, 1996 all incorporated herein by reference. Additional film forming polymers suitable for use herein include the water-insoluble polymer materials in aqueous emulsion and water soluble film forming polymers described in PCT publication No. WO98/18431, published on May 7, 1998 incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO_{(4-n)/2,} where n is a value between 1.0 and 1.50 and R is a methyl. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Michigan, KP545 from Shin-Etsu Chemical and G. E. 1170-002 from the General Electric Company. In the present invention, by having film forming polymer mainly in the second layer, the film forming polymer will exist in a higher concentration at a localized area, and thereby forming a film of higher film intensity when applied to the skin, compared to the remainder of the composition. Such concentrated area of high film intensity provides improved adhesion of active compositions to the skin. Namely, by providing the film forming polymer mainly in the second layer, the amount of film forming polymer included in the entire composition can be reduced, or if the same amount of film forming polymer is formulated in the second layer, an entire composition having improved adhesion is attained. In a preferred embodiment, the content level of film forming polymer in the second layer is from about 0.5% to about 20%, preferably from about 0.5% to about 10%, more preferably from about 1% to about 8%.

Soft Focus Agent (1) Soft Focus Powder

Soft focus powder is a pigment that is particularly effective in providing a soft focus effect to the skin, namely natural finish yet having good coverage for minimizing the appearance of skin troubles, when incorporated in a defined amount. Specifically, the soft focus powder herein must meet two parameters for providing such an effect. First, both the Total Luminous Transmittance (Tt) and Diffuse Luminous Transmittance (Td) of the pigment are relatively high. The soft focus powders have a Total Luminous Transmittance (Tt) of from about 40 to about 94 and a Diffuse Luminous Transmittance (Td) of from about 28 to about 38. Without being bound by theory, it is believed that, by having such high Tt and Td values, the soft focus powders exhibit a high transparency, thereby providing an overall natural finish. Second, the soft focus powders have a relatively high Haze value {(Td/Tt)×100} of from about 32 to about 95. Without being bound by theory, it is believed that, by having such high Haze value, the contrast between lighted area of the skin and shaded area of the skin (such as pores and wrinkles) is minimized for reducing the appearance of the trouble areas.

Total Luminous Transmittance (Tt), Diffuse Luminous Transmittance (Td), and Haze value {(Td/Tt)×100} can be measured and calculated by the artisan by reference to ASTM D 1003-00 “Standard Test Method for Haze and Luminous Transmittance of Transparent Plastics”. Although the pigments herein are not plastics, the same principles of this specific standard test can be applied.

The soft focus powder useful herein includes polymethyl/methacrylate(PMMA), silica, hybrid pigments such as alumina treated mica, titanium dioxide treated talc, titanium dioxide treated mica, vinyl dimethicone/methicone silsesquioxane crosspolymer, alumina, barium sulfate and synthetic mica. Commercially available soft focus powder useful herein includes the alumina treated mica with its Total Luminous Transmittance (Tt) is about 87, Diffuse Luminous Transmittance (Td) is about 28, and Haze value {(Td/Tt)×100} is about 32 under trade name SA Excel Mica JP2 available in Miyoshi Kasei. When soft focus powder is formulated in a single layer product, the content level of soft focus powder shall be as high as 5% to achieve noticeable natural look effect, because other powders contained in the formulation, such as coverage titanium dioxide may overwhelm the soft focus powder effect. As used herein, coverage titanium dioxides are those having a particle size of from about 200 nm to about 500 nm. If the particle size is out of this range, the titanium dioxide may not provide enough coverage as a cosmetic material. In the present invention, by formulating soft focus powders mainly in the second layer and coverage titanium dioxide in the first layer, and providing the first and second layers in a manner such that they can be simultaneously applied on the skin, the skin care product of the present invention can provide satisfied natural look effect with lower level of soft focus powder. As a result, the cost of the product can be decreased and a formulator will have more flexibility in the product formulation. In a preferred example, the content level of soft focus powders in the second layer is from about 2% to about 25%, more preferably from about 5% to about 20% based on the composition of the second layer. When calculated based on the total weight of the first layer and the second layer, the preferred content level of soft focus powders is from about 0.5% to about 4%, more preferably from about 2% to about 4%.

Soft Focus Agent (2) Silicone Elastomer

Soft focus silicone elastomer is crosslinked siloxane elastomer that is particularly effective in providing a soft focus effect to the skin, namely natural finish yet having good coverage for minimizing the appearance of skin troubles, when incorporated in a defined amount. Specifically, silicone elastomers have lower matte level compared with other silicone oils. Matte level is a parameter reflecting soft focus effect, i.e. natural finish of a cosmetic material. The lower the matte level is, the better natural finish the material can provide. Matte level of silicone elastomers used in the present application is less than about 40. Matte level can be measured by the PG-1M gloss meter (Incidence angle/Reflection angle: 60/60°) made by Nihon Denshoku Kogyo. Commercially available silicone elastomer useful in the present application includes a silicone elastomer having the tradename KSG-16 available from Shinetsu, which has a matte level of about 37.

Suitable silicone elastomers useful herein can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. Nos 4,970,252, 5,760,116, and 5,654,362. Additional crosslinked organopolysiloxane elastomers useful in the present invention are disclosed in Japanese Patent Application JP 61-18708, assigned to Pola Kasei Kogyo KK. Commercially available elastomers preferred for use herein are Dow Corning's 9040 silicone elastomer blend, Shin Etsu's KSG-21, and mixtures thereof.

When silicone elastomer is formulated in a single layer, the content level of silicone elastomer shall be as high as 10% to achieve noticeable natural look effect, because other powders in the formulation, such as coverage titanium dioxide may overwhelm the soft focus effect of silicone elastomer. However, in the present invention, by formulating a silicone elastomer mainly in the second layer and coverage titanium dioxide in the first layer, and providing the first and second layers in a manner such that they can be simultaneously applied on the skin, the skin care product of the present invention can provide satisfied natural look effect with lower level of soft focus powder. As a result, the cost of the product can be decreased and a formulator will have more flexibility in the product formulation. And also, spreadability performance can be improved. In a preferred example, the content level of silicone elastomer presented as a mixture in a solvent comprising 25% active level, such as KSG-16 in the second layer is from about 0.5% to about 40%, preferably from about 2% to about 30%. When calculated based on the total weight of the first layer and the second layer, the preferred content level of silicone elastomer is from about 0.5% to about 10%, more preferably from about 1% to about 7%.

Antiperspirant Agent

For an antiperspirant composition, the composition of the present invention may comprise a safe and effective amount of an antiperspirant active agent. A wide variety of conventional antiperspirant active agent are suitable for use herein, such as aluminum/zirconium astringent complexes including aluminum halides, aluminum hydroxy-halides, zirconyl oxyhalides, zirconyl hydroxy-halides; and ZAG complexes such as aluminium zirconium trichlorohydrex gly.

Volatile Silicone Oil

The composition of each layer of the present invention comprises volatile silicone oil. In a preferred cosmetic foundation embodiment, when the first layer is a water-in-oil emulsion, the composition of the first layer comprises from about 10% to about 50%, more preferably from about 15% to about 35% of volatile silicone oil; when the first layer is an oil-in-water emulsion, the composition of the first layer comprises from about 1% to about 30%, more preferably from about 5% to about 20% of volatile silicone oil; and the oil dispersion composition of the second layer comprises from about 10% to about 80%, more preferably from about 20% to about 70% of volatile silicone oil. Without being bound by theory, the species and levels of the volatile silicone oil herein is believed to provide improved refreshing and light feeling to the skin, without necessarily leaving a dried feeling to the skin.

The volatile silicone oil useful herein are selected from those having a boiling point of from about 60 to about 260° C., preferably those having from 2 to 7 silicon atoms.

The volatile silicone oils useful herein include polyalkyl or polyaryl siloxanes with the following structure (I): 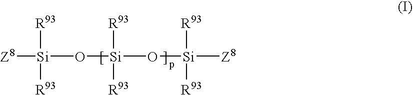
wherein R⁹³ is independently alkyl or aryl, and p is an integer from about 0 to about 5. Z⁸ represents groups which block the ends of the silicone chains. Preferably, R⁹³ includes methyl, ethyl, propyl, phenyl, methylphenyl and phenylmethyl, Z⁸ includes hydroxy, methyl, methoxy, ethoxy, propoxy, and aryloxy. More preferably, R⁹³ and Z⁸ are methyl. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula: 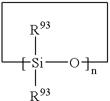
wherein R⁹³ is independently alkyl or aryl, and n is an integer of from 3 to 7.

Preferably, R⁹³ includes methyl, ethyl, propyl, phenyl, methylphenyl and phenylmethyl. More preferably, R⁹³ is methyl. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane under the tradename SH244, decamethylcyclopentasiloxane under the tradename DC245 and SH245, and dodeamethylcyclohexasiloxane under the tradename DC246; all available from Dow Coming.

Non-Volatile Oil

The composition of each layer of the present invention comprises non-volatile oil. In a preferred cosmetic foundation embodiment, when the first layer is a water-in-oil emulsion, the composition of the first layer comprises from about 0.5% to about 20%, more preferably from about 1% to about 15% of non-volatile oil; when the first layer is an oil-in-water emulsion, the composition of the first layer comprises from about 1% to about 20%, more preferably from about 5% to about 15% of non-volatile oil; and the oil dispersion composition of the second layer comprises from about 1% to about 40%, more preferably from about 5% to about 25% of non-volatile oil. Without being bound by theory, the species and levels of the non-volatile oil herein is believed to provide improved smoothness to the skin, and also alleviate dry feeling of the skin.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I) 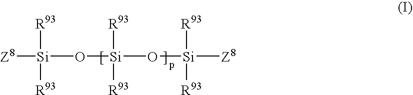
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ includes hydroxy, methyl, methoxy, ethoxy, propoxy, and aryloxy. The two R⁹³ on the silicon atom may represent the same group or different groups. Preferably, the two R⁹³ represents the same group. Suitable R⁹³ includes methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid, Shin-Etsu Chemical Co., Ltd. as KF-56.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

SOLID WAX

The water-in-oil emulsion and oil dispersion layer of the present invention comprises solid wax. In a preferred cosmetic foundation embodiment, when the first layer is a water-in-oil emulsion, the composition comprises from about 1% to about 10%, more preferably from about 2% to about 5% of solid wax, and the oil dispersion of the second layer comprises from about 1% to about 10%, more preferably from about 2% to about 7% of solid wax. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite was, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

LIPOPHILIC SURFACTANT

When the first layer of the present invention is a water-in-oil emulsion, composition of the first layer comprises a lipophilic surfactant, preferably by weight of the composition of the first layer at from about 0.5% to about 5%, more preferably from about 1% to about 4%. The lipophilic surfactant used herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

Without being bound by theory, the species and levels of the lipophilic surfactant herein are believed to provide a stable water-in-oil emulsion in view of the other components of the present invention.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), and (iii) as shown below, and mixtures thereof.
(i) dimethicone copolyols having the formula: 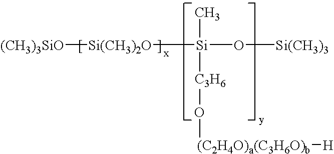
wherein x is an integer from 5 to 100, y is an integer from 1 to 50, a is zero or greater, b is zero or greater, the average sum of a+b is 1-100.
(ii) dimethicone copolyols having the formula: 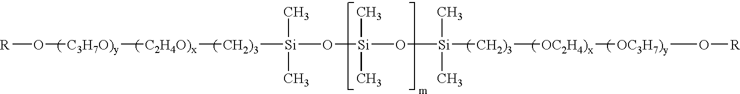
wherein R is selected from the group consisting of hydrogen, methyl, and combinations thereof, m is an integer from 5 to 100, x is independently zero or greater, y is independently zero or greater, the sum of x+y is 1-100.
(iii) branched polyether-polydiorganosiloxane emulsifiers herein having the formula: 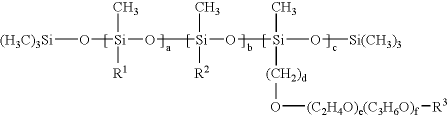
wherein R¹ is an alkyl group having from about 1 to about 20 carbons; R² is 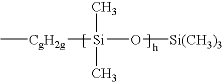
wherein g is from about 1 to about 5, and h is from about 5 to about 20; R³ is H or an alkyl having from about 1 to about 5 carbons; e is from about 5 to about 20; f is from about 0 to about 10; a is from about 20 to about 100; b is from about 1 to about 15; c is from about 1 to about 15; and d is from about 1 to about 5.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF-6028 available from ShinEtsu Chemical.

In a preferred embodiment, the lipophilic surfactant is a mixture of at least one ester-type surfactant and at least one silicone-type surfactant to provide a stable emulsion for the other essential components of the present invention.

Water

The composition of the first layer of the present invention comprises water in an amount sufficient to provide a discontinuous or continuous aqueous phase. In a preferred cosmetic foundation embodiment, when the first layer is a water-in-oil emulsion, the first layer composition comprises an amount of water such that the total of volatile silicone oil and water is more than about 40% of the composition, more preferably from about 10% to about 35% of water; and when the first layer is an oil-in-water emulsion, the composition of the first layer of the present invention comprises from about 20% to about 60%, more preferably from about 30% to about 50% of water. Without being bound by theory, the amount of water herein is believed to provide improved refreshing and light feeling to the skin, without necessarily leaving a dried feeling to the skin. Further, this amount of water allows the inclusion of optional water-soluble skin active agents as described below.

In the present invention, deionized water is typically used. Water from natural sources including mineral cations can also be used, depending on the desired characteristic of the product.

Pigment Powder Component

The composition of the first layer of the present invention comprises pigment powder component. In a preferred cosmetic foundation embodiment, when the first layer is a water-in-oil emulsion, the composition of the first layer comprises from about 5% to about 45%, more preferably from about 15% to about 30% of powder component; and when the first layer is an oil-in-water emulsion, the composition of the first layer comprises from about 5% to about 40%, more preferably from about 10% to about 30% of powder component. The oil dispersion layer of the present invention optionally comprises a pigment powder, when present, the oil dispersion composition comprises from about 1% to about 70%, more preferably from about 5% to about 50% of powder component. The pigment powders used herein are typically hydrophobic in nature, or hydrophobically treated for water-in-oil emulsion and oil dispersion, and hydrophilic in nature or non-hydrophobically treated for oil-in-water emulsion. By keeping the level of pigment component low, the entire composition maintains flexibility to accommodate other components which provide spreadability, moisturization, and fresh and light feel. The species and levels of the pigments are selected to provide, for example, shade, coverage, good wear performance, and stability in the composition.

Pigment powders useful for the present invention can be inorganic and organic powders such as talc, mica, sericite, silica, magnesium silicate, synthetic fluorphlogopite, calcium silicate, aluminum silicate, bentonite and montmorillonite; pearl pigments such as alumina, barium sulfate, calcium secondary phosphate, calcium carbonate, coverage titanium oxide, finely divided titanium oxide, zirconium oxide, normal particle size zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as Methicone, Dimethicone, and perfluoroalkylsilane; fatty material such as stearic acid and disodium hydrogenated glutamate; metal soap such as aluminium dimyristate; aluminium hydrogenated tallow glutamate, hydrogenated lecithin, lauroyl lysine, aluminium salt of perfluoroalkyl phosphate, and aluminium hydroxide as to reduce the activity for titanium dioxide, and mixtures thereof.

Commercially available hydrophobic pigment powder components include coverage titanium dioxide, such as titanium dioxide and talc and methicone: SI-T-CR-50Z available, titanium dioxide and methicone: SI-Titanium Dioxide IS, titanium dioxide and dimethicone: SA-Titanium Dioxide CR-50, titanium dioxide and methicone: SI-FTL-300 and titanium dioxide .and dimethicone and disodium hydrogenated glutamate: SA/NAI-TR-10, all of them are available from Miyoshi Kasei, iron oxide and cyclopentasiloxane and disodium hydrogenated glutamate: SA/NAI-Y-10/D5(70%)/SA/NAI-R-10/D5(65%) /SA/NAI-B-10/D5(75%) available from Miyoshi Kasei, iron oxide and disodium hydrogenated glutamate: SA/NAI-Y-10/SA/NAI-R-10/SA/NAI-B-10 available from Miyoshi Kasei, iron oxide and methicone: SI Mapico Yellow Light Lemon XLO/SI Pure Red Iron Oxide R-1599/SI Pure Red Iron Oxide R-3098/SI Pure Red Iron Oxide R-4098/SI Black Iron Oxide No.247 available from Daito Kasei, alumina and titanium dioxide and methicone: SI-LTSG30AFLAKE H (5%) LHC available from Miyoshi Kasei, talc and methicone: SI-Talc CT-20 available from Miyoshi Kasei, talc and methicone: SI-Talc JA13R LHC available from Miyoshi Kasei, mica and methicone: SI Mica available from Miyoshi Kasei, silica and dimethicone: SA-SB-300 available from Miyoshi Kasei, mica and methicone: SI Sericite available from Miyoshi Kasei, mica and dimethicone: SA Sericite available from Miyoshi Kasei, mica and C9-15 fluoroalcol phosphates and triethoxy caprylylsilane: FOTS-52 Sericite FSE available from Daito Kasei, talc and C9-15 fluoroalcol phosphates and triethoxy caprylylsilane: FOTS-52 Talc JA-13R available from Daito Kasei, boron nitride and methicone: SI02 Boron Nitride SHP-6 available from Daito Kasei, boron nitride and C9-15 fluoroalcol phosphates and triethoxy caprylylsilane: FOTS-52 Boron Nitride available from Daito Kasei, mica and titanium dioxide and methicone: SI Sericite TI-2 available from Miyoshi Kasei, mica and titanium dioxide and methicone: SI Mica TI-2 available from Miyoshi Kasei, talc and titanium dioxide and methicone: SI Talc TI-2 available from Miyoshi Kasei, lauroyl lysine: AMIHOPE LL available from Ajinomoto, synthetic fluorphlogopite and methicone: PDM-5L(S) / PDM-10L(S)/PDM-20L(S)/PDM-40L(S) available from Topy Industries,

Commercially available hydrophilic pigment components include coverage titanium dioxide, such as Titanium dioxide CR-50 available from Ishihara Techno Corporation, mica: Mica Y-3000 available from Yamaguchi Mica, talc: Talc JA13R available from Asada Milling, silica: MK-30 available from Fuji Silysia, iron oxides available from Titan Kogyo, boron nitride: Boron Nitride SHP-6 available from Mizushima Ferroalloy, barium sulfate: Pletelet Barium sulfate H, HF, HG, HL, HM, HP available from Sakai Chemical Industry.

Hydrophilic Surfactant

In a preferred cosmetic foundation embodiment, when the first layer is an oil-in-water emulsion, the composition of the first layer comprises hydrophilic surfactant at the level of from about 0.1% to about 4%, more preferably from about 0.3% to about 2%.

A wide variety of hydrophilic surfactant can be employed herein. Known or conventional hydrophilic surfactant can be used in the composition, provided that the selected hydrophilic surfactant is chemically and physically compatible with essential components of the composition, and provides the desired dispersion characteristics.

Non-limiting examples of hydrophilic surfactant useful herein are various non-ionic and anionic hydrophilic surfactant such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Non-limiting examples of other hydrophilic surfactant for use herein include: polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, and mixtures thereof.

Polyoxyalkylene hydrogenated castor oils useful herein include, for example, polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil.

Polyglycerin alkyl esters having the C10-20 of alkylsubstitute useful herein include, for example, those having 6-10 moles of glycerin units, such as polyglyceryl-6 laurate, polyglyceryl-10 laurate, and polyglyceryl-10 stearate.

Polysorbates useful herein include, for example, those having 20-80 moles of ethylene oxides, such as polysorbate-20, polyborbate-40, polysorbate-60, and polysorbate-80.

Polyethylene sterols and polyethylene hydrogenated sterols useful herein include, for example, those having 10-30moles of ethylene oxides, such as polyethylene (10) phytosterol, polyethylene (30) phytosterol, and polyethylene (20) cholesterol.

Among the above nonionic surfactants, preferred are polysorbates, and more preferred are polysorbate-20, polysorbate-40, and mixtures thereof.

Commercially available hydrophilic surfactant includes glyceryl stearate: Arlacel 161 available from Uniqema.

Fatty Compounds and Fatty Acid Salts

The oil-in-water emulsion composition of the present invention comprises fatty compounds or fatty acid salts. In a cosmetics foundation embodiment, preferably, the oil-in-water emulsion composition comprises from about 1% to about 15%, more preferably from about 4% to about 10% of fatty compounds or fatty acid salt.

Fatty compounds and fatty acid salts useful herein include stearic acid (e.g., stearic acid available from Kao), staric acid sodium salt, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

Additional Components

The compositions hereof may further contain additional components such as those conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Humectant

The composition of the present invention may further comprise a humectant by weight of the entire composition at from about 1% to about 15%, preferably 2% to about 7%.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

Skin Active Agent

The compositions of the present invention may comprise a safe and effective amount of a skin active agent. The term “skin active agent” as used herein, means an active ingredient which provides a cosmetic and/or therapeutic effect to the area of application on the skin, hair, or nails. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% of at least one skin active agent.

The type and amount of skin active agents are selected so that the inclusion of a specific agent does not affect the stability of the composition. For example, hydrophilic agents may be incorporated in an amount soluble in the aqueous phase, while lipophilic agents may be incorporated in an amount soluble in the oil phase.

Skin lightening agents useful herein refer to active ingredients that improve hyperpigmentation as compared to pre-treatment. Useful skin lightening agents herein include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include ascorbic acid per se in the L-form, sodium, potassium, lithium, calcium, magnesium, barium, ammonium and protamine salts of ascorbic acid, and derivatives thereof. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Vitamin B₃ compounds useful herein include, for example, those having the formula: 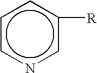
wherein R is -CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly Industries Inc.

Other hydrophobic skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, SMATVECTOR UV and Magnesium Ascorbyl Phosphate in Hyaluronic Filling Sphere available from COLETICA ,and mixtures thereof.

Other skin active agents useful herein include those selected from the group consisting of N-acetyl D-glucosamine, panthenol (e.g., DL panthenol available from Alps Pharmaceutical Inc.), tocopheryl nicotinate, benzoyl peroxide, 3-hydroxy benzoic acid, flavonoids (e.g., flavanone, chalcone), famesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV Protection Powder

UV protection powder provides UV protection benefit in the composition. UV protection powder has a particle size of less than 100 nm, which size does not have coverage effects. The composition of each layer of the present invention may comprise from about 0% to about 20%, preferably from about 0.1% to about 10% of a UV protection powder, such as micronized titanium dioxide and micronized zinc oxide. The powder included in the pigment component herein is typically hydrophobic in nature, or hydrophobically treated.

Commercially available UV protection powder is titanium dioxide and methicone SI-TTO-S-3Z available from Miyoshi Kasei, titanium dioxide and dimethicone and aluminum hydroxide and stearic acid: SAST-UFTR-Z available from Miyoshi Kasei, titanium dioxide: Titanium dioxide TTO-S-3 available from Ishihara Techno Corporation Zinc oxide: Finex series available from Sakai Chemical Industry.

UV Absorbing Agent

The compositions of the present invention may comprise a safe and effective amount of a UV absorbing agent. A wide variety of conventional UV protecting agent are suitable for use herein, such as those described in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

Thickener

Useful for the present invention is a thickener. Thickeners can be used for solidifying water-in-oil, oil-in-water or oil dispersion form compositions. When used, the thickener is kept to about 15% of the composition.

The thickeners useful herein are selected from the group consisting of gelling agents, inorganic thickeners, and mixtures thereof. The amount and type of thickeners are selected according to the desired viscosity and characteristics of the product.

The gelling agents useful as thickeners of the present invention include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:
R2-NH—CO—(CH2)2CH—(NH—CO—R1)—CO—NH—R2
wherein R1 is an aliphatic hydrocarbon radical having from about 12 to about 22 carbon atoms, and R2 is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:
R1-CO—(CH2)10-CH—(OH)—(CH2)5-CH3
wherein R1 is R2 or NR2R3; and R2 and R3 are hydrogen, or an alkyl, aryl, or arylalkyl radical which is branched linear or cyclic and has from about 1 to about 22 carbon atoms; preferably, from about 1 to about 18 carbon atoms. R2 and R3 may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy- 1,2,3 -propane tributylamide, 1 -propene- 1,2,3 -triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Inorganic thickeners useful herein include hectorite, bentonite, montmorillonite, and bentone clays which have been modified to be compatible with oil. Preferably, the modification is quaternization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

Preparation of the Composition

The present invention also relates to a suitable process for making the composition of the present invention. While the present composition may be made by any process known in the art, the process herein is advantageous for manufacturing the present composition in an aesthetically appealing, yet cost effective manner.

The present process is particularly useful for the present composition wherein the first layer and the second layer each provide a viscosity of from about 100 mPas to about 10,000 mPas, preferably from about 300 mPas to about 300 mPas when brought to a temperature of between about 55° C. and about 90° C. The present process comprises the steps of:

(a) providing the first layer composition and the second layer composition in fluid state in isolated vessels;

(b) separately dispensing the first layer composition by a first nozzle and the second layer composition by a second nozzle into a same package while keeping the temperature of the first layer composition and second layer composition in the range of 55° C. to 90° C., preferably in the range of 60° C. to 75°; and

(c) allowing the transferred first layer and second layer to solidify in the package.

Each of the first and second layer compositions may be made by a method well known in the art. In a suitable preparation process for oil dispersion, the composition is made by the steps of:
1) dissolving the volatile silicone oil, non-volatile oil, lipophilic surfactant, slurry of pigments dispersed in oil, and any other hydrophobic material in liquid form at ambient temperature in a sealed tank to make a lipophilic mixture;
2) adding the remaining pigments and powders into such lipophilic mixture and dispersing with a homogenizer at about 20-30° C.;
3) heating and adding to the product of step 2) solid wax and any remaining hydrophobic material at about 80-85° C.; and
4) cooling the finally obtained oil dispersion to a temperature of about 60-80° C.

The obtained composition, which is still fluid at such temperature, is filled in an air-tight container and allowed to cool to room temperature typically using a cooling unit. In a suitable preparation process for making an oil-in-water emulsion for the first layer, the composition is made by the steps of:
1) dissolving the water, humectant, fatty acid salts and any other hydrophilic material in liquid form at about 80-85° C. in a sealed tank, to make a hydrophilic mixture;
2) adding the remaining pigments and powders into such hydrophilic mixture and dispersing with a homogenizer;
3) dissolving the volatile silicone oil, non-volatile oil, fatty compounds in oil, and any other hydrophobic material in liquid form at ambient temperature in a sealed tank, to make a lipophilic mixture;
4) adding the product of step 3) to the product of step 2) to effect an emulsification;
5) cooling the finally obtained emulsion to a temperature of about 60-80° C.

The obtained composition, which is still fluid at such temperature, is filled in an air-tight container and allowed to cool to room temperature typically using a cooling unit. In a suitable preparation process for making a water-in-oil emulsion for the first layer; the composition is made by the steps of:
1) dissolving the volatile silicone oil, non-volatile oil, lipophilic surfactant, slurry of pigments dispersed in oil, and any other hydrophobic material in liquid form at ambient temperature in a sealed tank, to make a lipophilic mixture;
2) adding the remaining pigments and powders into such lipophilic mixture and dispersing with a homogenizer at about 20-30° C.;
3) separate from 1) and 2), heating and dissolving in water, humectants and any other hydrophilic material at about 75-80° C., and then cooling to about 20-30° C.;
4) adding the product of step 3) to the product of step 2) to provide an emulsification;
5) heating and adding to the product of step 4) solid wax and any remaining hydrophobic material at about 80-85° C.; and
6) cooling the finally obtained emulsion to a temperature of about 60-80° C.
   The obtained composition, which is still fluid at such temperature, is filled in an air-tight container and allowed to cool to room temperature typically using a cooling unit

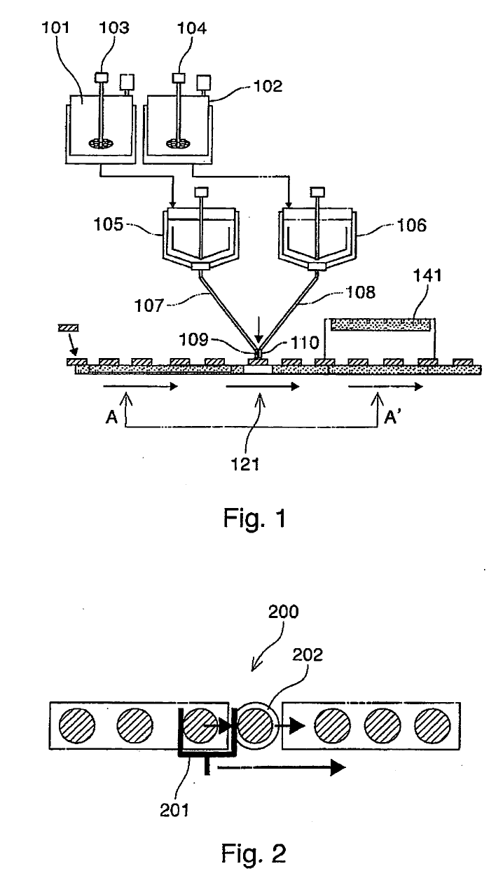

Referring to FIG. 1, the first and second layer compositions made according to the above steps are re-melted under 70° C. and deaerated in two isolated vessels 101 and 102. Such vessel is typically a tank that is equipped with appropriate mixing means 103 and 104 for mixing and homogenizing. Then, the deaerated bulk compositions are transferred into two separate filling hoppers 105 and 106, from where the first and second layer compositions in fluid state are delivered into pipes 107, 108 which are guided to a first nozzle 109 for the first layer, and a second nozzle 110 for the second layer. In a preferred embodiment, the second nozzle 110 is composed of two separate nozzles. The first and second nozzles terminate at a filling site 121. In the process of transferring and filling, heat-exchanging equipments are used to maintain the bulk composition temperature within the range of about 55° C. to about 90° C., preferably from about 60° C. to about 75° C.

Meanwhile, the reservoir part of the primary package for accommodating the present composition is brought to the filling site 121 by suitable means such as a moving belt conveyor 120. In the preferred foundation embodiment of the present invention, the reservoir part of the primary package is a pan made of metallic or plastic material. In the description hereafter, the reservoir part of the primary package is represented by, and referred to as a “pan”. Now referring to FIG. 2, the pan is brought to filling site 200 by means of, for example, a moving bar 201. The filling site 200 consists of a table 202 for placing the pan, and at which the primary package receives the first and second layer compositions in fluid state by the first nozzle and second nozzle. The table 202 may be moved or rotated so that a design is illustrated by the flow of the first and second layer compositions in fluid state. The terminating point of the first and second nozzle may also be moved or rotated. Depending on the combined movement of the table and nozzle termination points, various designing is possible. Here, it is advantageous to have the first and second layer compositions visibly distinct, such that the design is clear and distinct.

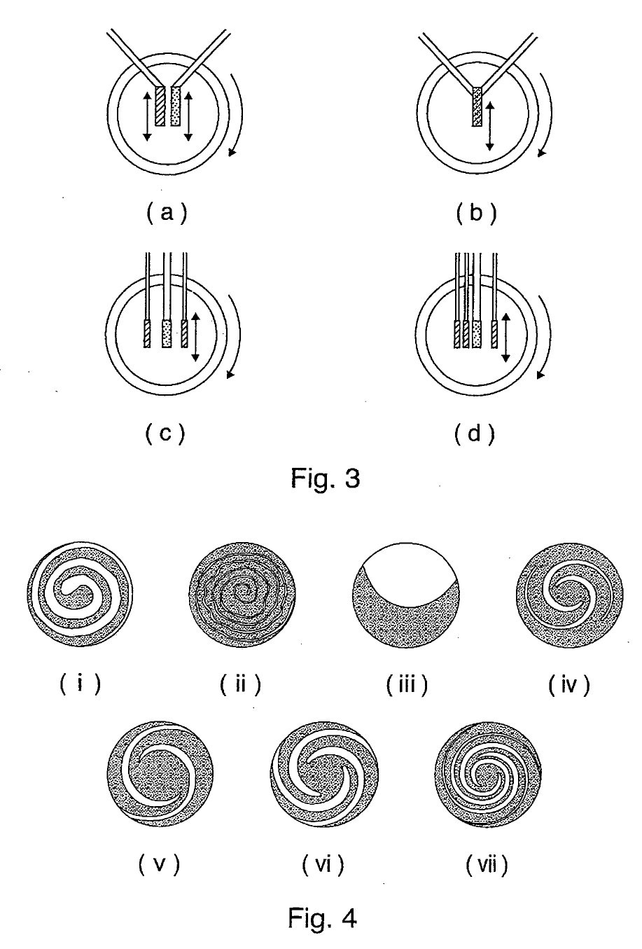

FIG. 4 shows embodiments of the resulting design made by such movement of the table and/or nozzle termination points upon filling. The design of (iii) may be made by having one nozzle stable, and the other nozzle moving in linear direction. The spiral design of (i) may be made by the first and second nozzles moving away from each other in linear direction, while the table is rotated as shown in FIG. 3(a). Another spiral design (iv) may be made in a similar manner, albeit by having one of the first or second nozzle separated into two branches, as shown in (c) of FIG. 3. Yet another spiral design (v) can be made by the same nozzle configuration shown in (c) of FIG. 3, albeit adjusting the filling speed and rotation speed of the table. Other spiral designs (vi) and (vii) of FIG. 4 can be made by the nozzle configuration as shown in (d) of FIG. 3, wherein one of the first or second nozzle is separated into three branches. The marble design of (ii) of FIG. 4 may be made by having the first nozzle and second nozzle jointed with each other immediately before the termination point, such as shown in (b) of FIG. 3. In such an embodiment, the temperature of the first and second layer compositions must be carefully controlled in between 60° C. and 75° C. such that the layers are not completely mixed with each other at the joint point, yet are fluid enough to flow.

Referring back to FIG. 1, the pan filled with the first and second layer compositions are sent to another moving belt conveyer, and moved through a cooling unit 141 for cooling and solidifying the composition. The obtained composition is then engaged with other parts of the primary package, such as the lid, and outer wall accommodating the pan.

Those compositions containing volatile components such as water, silicone oil, and others, are packaged in an air-tight container, such that the composition is not deteriorated during storage. In the preferred foundation embodiment of the present invention, the composition is placed in a compact housing an air tight container in which the composition is included. The compact may further contain a mirror and a concave tray for accommodating a sponge applicator.

Application of the Product

The multilayer product of the present invention can be applied on a consumer's skin by a finger, a sponge or a brush. Depending on how well consumers mix each layers before application, each layers of the product may be maintained to be separated, semi-mixed or mixed upon application on the skin. However, to achieve certain benefits of the present invention, such as oil control from oil absorbing agent, natural look from soft focus agent, it is preferred to apply the present product by paying off the product from the package by a finger, a sponge or a brush in one stroke, and then applying the product on skin. It is believed that by using this preferred application method, each layer of the product of the present invention will more or less maintain being separated from each other even upon applying on the skin, and therefore can achieve the intended benefits for skin.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### 1) Examples 1-5 (W/O Solid Emulsion Composition Formula of the First Layer)

The following make-up compositions are formed by the process described below herein:

| NO. | Components | Ex. 1-1 | Ex. 2-1 | Ex. 3-1 | Ex. 4-1 | Ex. 5-1 |
|---|---|---|---|---|---|---|
| 1 | Cyclopentasiloxane *1 | 25.90 | 25.90 | 25.90 | 25.90 | 25.90 |
| 2 | PEG-9 | 1.50 | 1.50 | 1.50 | 1.50 | 1.50 |
| | Polydimethylsiloxyethyl | | | | | |
| | Dimethicone *2 | | | | | |
| 3 | Tocopheryl Acetate *3 | 0.50 | 0.50 | 0.50 | 0.50 | 0.50 |
| 4 | Isotridecyl Isononanoate *4 | 2.00 | 2.00 | 2.00 | 2.00 | 2.00 |
| 5 | Sorbitan Monoisostearate *5 | 1.50 | 1.50 | 1.50 | 1.50 | 1.50 |
| 6 | Iron oxide and | 2.00 | 1.80 | 2.00 | 2.00 | 2.00 |
| | Cyclopentasiloxane and | | | | | |
| | Dimethicone and Disodium | | | | | |
| | Hydrogenated Glutamate *6 | | | | | |
| 7 | Titanium Dioxide and | — | — | — | — | 8.00 |
| | Dimethicone and Disodium | | | | | |
| | Hydrogenated Glutamate *7 | | | | | |
| 8 | Titanium Dioxide and Talc | 14.00 | 12.60 | 10.00 | 12.00 | — |
| | and Methicone *8 | | | | | |
| 9 | Alumina and Titanium | — | — | — | 2.00 | 3.00 |
| | Dioxide and Methicone *9 | | | | | |
| 10 | Titanium Dioxide and | — | — | 5.00 | — | — |
| | Methicone *10 | | | | | |
| 11 | Titanium Dioxide and | 3.00 | 3.00 | 3.00 | 3.00 | 5.00 |
| | Dimethicone and Aluminium | | | | | |
| | Hydroxide and Stearic Acid *11 | | | | | |
| 12 | Talc and Methicone *12 | 7.00 | 8.60 | 6.00 | 7.00 | 7.00 |
| 13 | Water | 29.00 | 29.00 | 30.00 | 28.00 | 30.00 |
| 14 | Niacinamide *13 | 4.00 | 4.00 | 3.00 | 3.00 | 4.00 |
| 15 | N-acetyl D-glucosamine | — | — | — | 2.00 | — |
| 16 | Preservative | 0.45 | 0.45 | 0.45 | 0.45 | 0.45 |
| 17 | Panthenol *14 | 0.25 | 0.25 | 0.25 | 0.25 | 0.25 |
| 18 | Glycerin *15 | — | — | — | 2.00 | 5.00 |
| 19 | Butylene Glycol *16 | 5.00 | 5.00 | 5.00 | 3.00 | — |
| 20 | Candelilla Wax *17 | 2.00 | 2.00 | 2.00 | 2.00 | 2.00 |
| 21 | Ceresin *18 | 1.90 | 1.90 | 1.90 | 1.90 | 1.90 |

| | | | | | |
|---|---|---|---|---|---|
| Total | 100.00 | 100.00 | 100.00 | 100.00 | 100.00 |
| Density (g/cm³) | 1.190 | 1.200 | 1.180 | 1.220 | 1.230 |
| Viscosity (mPas) | 730 | 700 | 950 | 820 | 580 |

Definitions of Components
*1 Cyclopentasiloxane: SH245 available from Dow Corning
*2 PEG-9 Polydimethylsiloxyethyl Dimethicone: KF-6028 available from Shin-Etsu Chemical Co., Ltd.
*3 Tocopheryl Acetate: DL-α-tocopheryl Acetate available from Eisai
*4 Isotridecyl isononanoate: Crodamol TN available from Croda
*5 Sorbitan isostearate: Crill 6 available from Croda
*6 Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*7 Titanium Dioxide and Dimethicone and Disodium Hydrogenated Glutamate: SA/NAI TR-10 from Miyoshi Kasei
*8 Titanium Dioxide and Talc and Methicone: SI-T-CR-50Z available from Miyoshi Kasei
*9 Alumina and Titanium Dioxide and Methicone: SI-LTSG30AFLAKEH(5%)LHC available from Miyoshi Kasei
*10 Titanium Dioxide and Methicone: SI-FTL-300 available from Miyoshi Kasei
*11 Titanium Dioxide and Dimethicone and Aluminium Hydroxide and Stearic acid: SAST-UFTR-Z available from Miyoshi Kasei
*12 Talc and Methicone: SI Talc JA13R LHC available from Miyoshi Kasei
*13 Niacinamide: Niacinamide available from Reilly Industries Inc.
*14 Panthenol: DL-Panthenol available from Alps Pharmaceutical Inc.
*15 Glycerin: Glycerin USP available from Asahi Denka
*16 Butylene Glycol: 1,3-Butylene Glycol available from Kyowa Hakko Kogyo
*17 Candelilla wax: Candelilla wax NC-1630 available from Cerarica Noda
*18 Ceresin: Ozokerite wax SP-1021 available from Strahl & Pitsh

Preparation Method

The W/O solid emulsion composition of the first layer of Examples 1-1 - 5-1 are prepared as follows:
1) Mixing components numbers 1 through 6 with suitable mixer until homogeneous to provide a silicone phase.
2) Mixing components numbers 7 through 12 with suitable mixer until homogeneous to provide a pigment mixture which is then pulverized using a pulverizer. And adding the pigment mixture into the silicone phase with suitable mixer until homogeneous.
3)solving components number 13 through 19 with suitable mixer until all components are dissolved to provide a water phase. Adding the water phase into the silicone phase and pigment mixture to provide an emulsion at room temperature using a homogenizer.
4) Adding components number 20 and 21 into the emulsion which is then heated to dissolve at 85° C. in a sealed tank.
5) Finally, filling the emulsion into an air-tight container and allowing cooling to room temperature using a cooling unit.

### 2) Examples 6-10 (O/W Solid Emulsion Formula for First Layer)

The following make-up compositions are formed by the process described herein:

| No. | Components | Ex. 6-1 | Ex. 7-1 | Ex. 8-1 | Ex. 9-1 | Ex. 10-1 |
|---|---|---|---|---|---|---|
| 1 | Cyclopentasiloxane *1 | 14.50 | 14.50 | 14.50 | 10.50 | 12.50 |
| 2 | Stearic Acid *2 | 1.20 | 1.20 | 1.20 | 1.20 | 1.20 |
| 3 | Glyceryl Stearate *3 | 1.00 | 1.00 | 1.00 | 1.00 | 1.00 |
| 4 | Tocopheryl Acetate *4 | 0.50 | 0.50 | 0.50 | 0.50 | 0.50 |
| 5 | Isotridecyl Isononanoate *5 | 2.00 | 2.00 | 2.00 | 2.00 | 2.00 |
| 6 | Phenyl Trimethicone *6 | 8.00 | 8.00 | 8.00 | 8.00 | 8.00 |
| 7 | Iron oxides | 2.00 | 2.00 | 2.00 | 2.00 | 2.00 |
| 8 | Titanium Dioxide *7 | 8.00 | 8.00 | 8.00 | 8.00 | 10.00 |
| 9 | Mica | 5.00 | 11.00 | 11.00 | 6.00 | 9.00 |
| 10 | Talc | 6.00 | — | — | 5.00 | — |
| 11 | Water | 38.00 | 38.00 | 38.00 | 40.00 | 38.00 |
| 12 | Stearic acid Sodium Salt | 6.50 | 6.50 | 6.50 | 6.50 | 6.50 |
| 13 | Triethanolamine | 0.10 | 0.10 | 0.10 | — | — |
| 14 | Potassium Hydroxide | — | — | — | 0.10 | 0.10 |
| 15 | Niacinamide *8 | 3.50 | 3.50 | 3.50 | 3.50 | 3.50 |
| 16 | Preservative | 0.45 | 0.45 | 0.45 | 0.45 | 0.45 |
| 17 | Panthenol *9 | 0.25 | 0.25 | 0.25 | 0.25 | 0.25 |
| 18 | Glycerin *10 | -— | — | 3.00 | 2.00 | — |
| 19 | Butylene Glycol *11 | 3.00 | 3.00 | — | 3.00 | 5.00 |

| | | | | | |
|---|---|---|---|---|---|
| Total | 100.00 | 100.00 | 100.00 | 100.00 | 100.00 |
| Density (g/cm³) | 1.150 | 1.140 | 1.160 | 1.190 | 1.170 |
| Viscosity (mPas) | 850 | 900 | 930 | 1000 | 980 |

Definitions of Components
*1 Cyclopentasiloxane: SH245 available from Dow Corning
*2 Stearic Acid: Stearic Acid 750 available from Kao
*3 Glyceryl Stearate: Arlacel 161 available from Uniqema
*4 Tocopheryl Acetate: DL-α-tocopheryl Acetate available from Eisai
*5 Isotridecyl isononanoate: Crodamol TN available from Croda
*6 Phenyl Trimethicone: KF-56 available from Shin-Etsu Chemical Co., Ltd.
*7 Titanium Dioxide: Titanium dioxide CR-50 available from Ishihara Techno Corporation
*8 Niacinamide: Niacinamide available from Reilly Industries Inc.
*9 Panthenol: DL-Panthenol available from Alps Pharmaceutical Inc.
*10 Glycerin: Glycerin USP available from Asahi Denka
*11 Butylene Glycol: 1,3-Butylene Glycol available from Kyowa Hakko Kogyo

Preparation Method
The make-up compositions of Examples 6-10 are prepared as follows:
1) Mixing components numbers 11 through 19 with suitable mixer and heat to dissolve at 75 degree C to provide a water phase.
2) Mixing components numbers 7 through 10 with suitable mixer until homogeneous to provide a pigment mixture which is then pulverized using a pulverizer. And adding the pigment mixture into the water phase with a suitable mixer until homogeneous.
3) Mixing components number I through 6 with suitable mixer and heating to dissolve at 80 degree C to provide an oil phase. And adding the oil phase into the water phase and pigment mixture to provide an emulsion using a homogenizer.
4) Finally, filling the emulsion into an air-tight container and allowing cooling to room temperature using a cooling unit.

### 3) Examples 1-10 (Oil Dispersion Formula for the Second Layer)

The Following make-up compositions are formed by the process described herein:

| | | Ex. 1-2 | Ex. 2-2 | Ex. 3-2 | Ex. 4-2 | Ex. 5-2 |
|---|---|---|---|---|---|---|
| NO. | Components | Ex. 6-2 | Ex. 7-2 | Ex. 8-2 | Ex. 9-2 | Ex. 10-2 |
| 1 | Cyclopentasiloxane *1 | 56.25 | 51.25 | 55.75 | 41.25 | 33.35 |
| 2 | PEG-9 | — | — | — | — | 1.00 |
| | Polydimethylsiloxyethyl | | | | | |
| | Dimethicone *2 | | | | | |
| 3 | Dimethicone and | 5.00 | — | 25.00 | — | — |
| | Dimethicone/Vinyl | | | | | |
| | Dimethicone Crosspolymer *3 | | | | | |
| 4 | Trimethylsiloxysilicate and | — | — | — | 15.00 | — |
| | Cyclopentasiloxane *4 | | | | | |
| 5 | Phenyl Trimethicone *5 | 5.00 | 5.00 | 2.00 | 5.00 | — |
| 6 | Isotridecyl Isononanoate *6 | 2.00 | 2.00 | 2.00 | 2.00 | 10.00 |
| 7 | Sorbitan Monoisostearate *7 | - | 3.00 | 1.00 | 3.00 | 3.00 |
| 8 | Iron oxide and | — | — | — | — | — |
| | Cyclopentasiloxane and | | | | | |
| | Dimethicone and Disodium | | | | | |
| | Hydrogenated Glutamate *8 | | | | | |
| 9 | Titanium Dioxide and | — | — | — | — | — |
| | Cyclopentasiloxane and | | | | | |
| | Dimethicone and Disodium | | | | | |
| | Hydrogenated Glutamate *9 | | | | | |
| 10 | Methyl Methacryate | 15.00 | 15.00 | — | 10.00 | — |
| | Crosspolymer and | | | | | |
| | Methicone *10 | | | | | |
| 11 | Silica and Methicone *11 | — | 5.00 | — | 5.00 | 10.00 |
| 12 | Vinyl Dimethicone/Methicone | 8.00 | — | — | — | — |
| | Silsesquioxane Crosspolymer *12 | | | | | |
| 13 | Mica and Zinc Oxide and | — | 10.00 | — | 5.00 | — |
| | Methicone and | | | | | |
| | Hydroxyapatite *13 | | | | | |
| 14 | Talc and Methicone *14 | 5.00 | — | — | 5.00 | 30.00 |
| 15 | Mica and Methicone *15 | — | 5.00 | 10.00 | 5.00 | 10.00 |
| 16 | Preservative | 0.25 | 0.25 | 0.25 | 0.25 | 0.25 |
| 17 | Candelilla Wax *16 | 1.80 | 1.80 | 2.00 | 1.80 | 1.20 |
| 18 | Ceresin *17 | 1.70 | 1.70 | 2.00 | 1.70 | 1.20 |

| | | | | | |
|---|---|---|---|---|---|
| Total | 100.00 | 100.00 | 100.00 | 100.00 | 100.00 |
| Density (g/cm3) | 1.110 | 1.120 | 1.050 | 1.180 | 1.270 |
| Viscosity (mPas) | 700 | 400 | 800 | 600 | 700 |

Definitions of Components
*1 Cyclopentasiloxane: SH245 available from Dow Corning
*2 PEG-9 Polydimethylsiloxyethyl Dimethicone: KF-6028 available from Shin-Etsu Chemical Co., Ltd.
*3 Dimethicone and Dimethicone/Vinyl Dimethicone Crosspolymer: KSG-16
available from Shin-Etsu Chemical Co., Ltd.
*4 Trimethylsiloxysilicate and Cyclopentasiloxane: Trimethilsiloxysilicate/Cyclomethicone D5 Blend available from GE Toshiba Silicones
*5 Phenyl Trimethicone: KF-56 available from Shin-Etsu Chemical Co., Ltd.
*6 Isotridecyl isononanoate: Crodamol TN available from Croda
*7 Sorbitan monoisostearate: Crill 6 available from Croda
*8 Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*9 Titanium Dioxide and Cyclopentasiloxane and Dimethicone and Disodium
Hydrogeriated Glutamate: SA/NAI-TR-10/D5(80%) available from Miyoshi Kasei
*10 Methyl Methacryate Crosspolymer and Methicone: SI-L-XC-F006Z available from Miyoshi Kasei
*11 Silica and Methicone: SI-SILDEX H-52 available from Miyoshi Kasei
*12 Vinyl Dimethicone/Methicone Silsesquioxane Crosspolymer: KSP-100 available from Shin-Etsu Chemical Co., Ltd.
*13 Mica and Zinc Oxide and Methicone and Hydroxyapatite: SI-PLV-20 available from Miyoshi Kasei
*14 Talc and Methicone: SI Talc CT-20 available from Miyoshi Kasei
*15 Mica and Methicone: SI Mica available from Miyoshi Kasei
*16 Candelilla wax: Candelilla wax NC-1630 available from Cerarica Noda
*17 Ceresin: Ozokerite wax SP-1021 available from Strahl & Pitsh

Preparation Method
The make-up compositions of Examples 1-2 - 10-2 are prepared as follows:
1) Mixing components numbers 1 through 7 with suitable mixer until homogeneous to provide a silicone phase.
2) Mixing components numbers 8 through 15 with suitable mixer until homogeneous to provide a pigment mixture. The pigment mixture is then pulverized using a pulverizer. Adding the pigment mixture into the silicone phase with a suitable mixer until homogeneous.
3) Adding components number 16 through 18 into the emulsion which is heated to dissolve at 85° C. in a sealed tank.
4) Finally, filling the emulsion into an air-tight container and allowing cooling to room temperature using a cooling unit.

Five different dual-layer foundation products comprising a first water-in-oil emulsion layer and a second oil dispersion layer are made by combining, at the weight ratio of 9:1 to 1:9 of the first layer composition, 1-1 to 5-1 and the corresponding second layer composition, 1-2 to 5-2 of Examples 1-5 and using the preparation method described above. Another five different dual-layer foundation products comprising a first oil-in-water emulsion layer and a second oil dispersion layer are made by combining, at the weight ratio of 9:1 to 1:9 of the first layer composition, 6-1 to 10-1 and the corresponding second layer composition, 6-2 to 10-2 of Examples 6-10 and using the preparation method described above. Specifically, the preparation process includes the steps of (a) remelting and deaerating the first layer composition of Example 1-1 to 10-1 and the second layer composition of Example 1-2 to 10-2 in two isolated vessels; (b) separately dispensing the first layer composition by a first nozzle and the second layer composition by a second nozzle into a same package while keeping the temperature of the first layer composition and second layer composition between 60° C. and 75° C.; and (c) allowing the transferred first layer and second layer to solidify in the package. The dual-layer foundation products of the present invention not only have a more attractive aesthetic look, but also provide a variety of skin benefits. For example, Example 1 and 6 can provide natural look with oily shine control benefit by comprising methyl methacryate crosspolymer and methicone: SI-L-XC-F006Z available from Miyoshi Kasei in the second layer, Example 2 and 7 can provide natural look with oily shine control benefit by comprising methyl methacryate crosspolymer and methicone: SI-L-XC-F006Z available from Miyoshi Kasei, silica and methicone: SI-Sildex H-52 available from Miyoshi Kasei and mica and zinc oxide and methicone and hydroxyapatite: SI-PLV-20 available from Miyoshi Kasei in the second layer, Example 3 and 8 can provide natural look by comprising dimethicone and dimethicone/vinyl dimethicone crosspolymer: KSG-16 available from Shin-Etsu Chemical Co., Ltd. in the second layer, Example 4 and 9 can provide oil shine control, long wear and natural look benefit by comprising trimethylsiloxysilicate and cyclopentasiloxane: trimethylsiloxysilicate D5 Blend available from GE Toshiba Silicones, methyl methacryate crosspolymer and methicone: SI-L-XC-F006Z available from Miyoshi Kasei, silica and methicone: SI-Sildex H-52 available from Miyoshi Kasei and mica and zinc oxide and methicone and hydroxyapatite: SI-PLV-20 available from Miyoshi Kasei in the second layer and Example 5 and 10 can provide oily shine control benefit by comprising silica and methicone: SI-Sildex H-52 available from Miyoshi Kasei in the second layer.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid skin care composition comprising:
(a) a first layer which is solid at 45° C. and which is a water-in-oil emulsion or an oil-in-water emulsion; and
(b) a second layer which is solid at 45° C. and which is an oil dispersion comprising a benefit agent;
wherein the first layer and the second layer are provided in the same package in a manner such that the first layer and the second layer can be simultaneously applied.

2. The composition of claim 1 wherein the first layer and the second layer are visibly distinct.

3. The composition of claim 2 wherein at least one of the first layer and second layer comprises a colorant to make the layers visibly distinct.

4. The composition of claim 1, wherein the weight ratio of the first layer to the second layer is from about 1:99 to about 99:1.

5. The composition of claim 1 wherein the first layer and the second layer each provide a viscosity of from about 100 mPas to about 300 mPas when brought to a temperature of between about 55° C. and about 90° C.

6. The composition of claim 1, wherein the viscosity difference and density difference between the compositions of the first layer and the second layer are within the area defined by the points of a(0.16 g/cm³, −1600 mPas), b(0.16 g/cm³, 600 mPas), c(−0.16 g/cm³, −600 mPas) and d(−0.16 g/cm³, 1600 mPas) as shown in the diagram of FIG. 5.

7. The composition of claim 1 wherein the second layer is an oil dispersion composition comprising:
(a) a volatile silicone oil;
(b) a non-volatile oil;
(c) a solid wax;
(d) optionally, a pigment powder; and
(e) a benefit agent.

8. The composition of claim 7 wherein the first layer composition is water-in-oil emulsion comprising:
(a) a volatile silicone oil;
(b) a non-volatile oil;
(c) a pigment powder;
(d) a solid wax;
(e) a lipophilic surfactant; and
(f) water.

9. The composition of claim 7 wherein the first layer composition is an oil-in-water emulsion comprising:
(a) water;
(b) a hydrophilic surfactant;
(c) a pigment powder;
(d) a non-volatile oil;
(e) a volatile silicone oil; and
(f) a fatty compound or fatty acid salts

10. The composition of claim 8 wherein the composition is a cosmetic foundation and wherein the benefit agent of the second layer is selected from the group consisting of an oil absorbing powder, sebum solidifying powder, film forming polymer, soft focus agents and mixtures thereof.

11. The composition of claim 9 wherein the composition is a cosmetic foundation and wherein the benefit agent of the second layer is selected from the group consisting of an oil absorbing powder, sebum solidifying powder, film forming polymer, soft focus agents and mixtures thereof.

12. The composition of claim 1 wherein the composition is an antiperspirant product, wherein the benefit agent is an antiperspirant active agent.

13. A method of manufacturing the composition of claim 5 comprising the steps of:
(a) providing the first layer composition and the second layer composition in fluid state in isolated vessels;
(b) separately dispensing the first layer composition by a first nozzle and the second layer composition by a second nozzle into a same package while keeping the temperature of the first layer composition and the second layer composition between about 55° C. and about 90° C.; and
(c) allowing the transferred first layer and second layer to solidify in the package.

14. The method of claim 12, wherein upon filling of the first layer and second layer into the package, the package is rotated.

15. The method of claim 12, wherein the second nozzle is composed of two separate nozzles.

16. The method of claim 12, wherein the second nozzle is composed of three separate nozzles.
